(19) United States
(12) Patent Application Publication
(10) Pub. No.: US 2022/0183961 A1
(43) Pub. Date: Jun. 16, 2022

(54) PHYSICAL SUNSCREEN COMPRISING HYDROXYAPATITE OR MODIFIED HYDROXYAPATITE OBTAINED FROM FISHERIES AND AQUACULTURE WASTE, PROCESS FOR ITS PRODUCTION AND PHOTOPROTECTIVE COMPOSITIONS COMPRISING IT

(71) Applicant: CONSIGLIO NAZIONALE DELLE RICERCHE, Roma (IT)

(72) Inventors: Michele IAFISCO, Castel Maggiore (IT); Alessio ADAMIANO, Bologna (IT); Clara PICCIRILLO, Lecce (IT)

(73) Assignee: CONSIGLIO NAZIONALE DELLE RICERCHE, Roma (IT)

(21) Appl. No.: 17/599,168

(22) PCT Filed: Mar. 27, 2020
(86) PCT No.: PCT/EP2020/058694

§ 371 Date: Sep. 28, 2021

(30) Foreign Application Priority Data
Mar. 28, 2019 (IT) 102019000004673

(51) Int. Cl. A61K 8/98; A61Q 17/04; A61K 8/02; A61K 8/24; A61K 8/27; A61K 8/29; A01G 7/06

(52) U.S. Cl. CPC A61K 8/987; A61Q 17/04; A61K 8/022; A01G 7/06; A61K 8/27; A61K 8/29; A61K 8/24

(57) ABSTRACT

It is described a process, starting from fishbones, for the production of a material having properties of physical type solar filter and photoprotective boosting agent, formed by particles of hydroxyapatite or hydroxyapatite substituted with metal ions, optionally mixed with calcium triphosphate and with metal oxides. Are also described the material obtained through the process and cosmetic or plant sunscreen compositions that comprise said material.

### FIELD OF THE INVENTION

The present invention relates to a process for the production, starting from fish waste, of a material that has the function of a physical type solar filter and photoprotective boosting agent, formed by particles of hydroxyapatite or hydroxyapatite modified with ions of elements different from those of pure hydroxyapatite, optionally mixed with tricalcium phosphate and with oxides of said elements. The invention also refers to the material indicated above and to a cosmetic composition comprising it. Moreover, the material can be used as active ingredient in formulations for the photoprotection of plants.

### STATE OF THE ART

It is known that solar radiation, and in particular the ultraviolet component (UV) of the spectrum of this radiation, is responsible for photochemical degradations of various kinds. In humans, in particular, acute and chronic exposure to UV rays can lead to skin rash, burns, photo-aging, photo-immunosuppression, and potentially to the onset of skin cancer (photocarcinogenesis). In plants, an excessive amount of UV radiation can determine leaves and fruit bleaching, decreased carbon dioxide fixation and oxygen evolution, reduction in dry weight, starch and chlorophyll content, marked reduction of plants growth and potentially severe oxidative stress, as described for instance in patent application WO 2009/064450 A1.

The UV radiation includes the part of the wavelength spectrum between about 100 and 400 nm, which are further divided into UVC (100-280 nm), UVB (280-320 nm) and UVA (320-400 nm). Exposure to UVC radiation is of little practical interest, as wavelengths below 280 nm are absorbed by atmospheric ozone and do not reach the earth's surface, while exposure to UVA and UVB is considered inevitable.

To prevent or mitigate the negative effects of UV exposure, so-called sunscreens are used, namely, fluid compositions that can be distributed on the part to be protected and formed by a vehicle in which one or more components are dispersed, generally referred to as solar filters, which can reduce the amount of UV radiation that reaches the part itself.

Solar filters are divided into two main classes: chemical or organic filters, in which the active components in photoprotection are organic molecules capable of absorbing UV rays, and physical or inorganic UV filters, including physical barriers that reflect radiation.

Among the photoprotective components of chemical type, the one which is most widely used is the compound 1-(4-methoxyphenyl)-3-(4-tert-butylphenyl)-propane-1,3-dione, commonly referred to with the name avobenzone, while among the most commonly used physical compounds, TiO₂ and ZnO can be mentioned in particular.

However, the solar filters currently on the market are not exempt from critical issues.

A problem observed with chemical filters is their photocatalytic activity, which can lead to their photochemical degradation and/or to the degradation of other components in sunscreen formulations, and to the generation of free radicals and other reactive species that may themselves be the cause of some of the health problems associated with UV exposure;

in this regard, see for example the paper “Current Sunscreen Controversies: A Critical Review” M. E. Burnett et al., Photodermatology Photoimmunology and Photomedicine, 2011, 27(6): 58-67.

As regards the physical filters, due to their increasing use, increasing concentrations of TiO₂ and ZnO nanoparticles in the environment (especially in coastal waters) have recently been detected, which exert ecotoxic effects in both aquatic and terrestrial species; these nanoparticles also have negative effects due to their photocatalytic activity; see, for example, the paper “Ecotoxicity of manufactured ZnO nanoparticles—A review” H. Ma et al., Environmental Pollution, 2013, 172, 76-85.

Another problem related to the use of inorganic nanoparticle based UV filters regards the epidermal penetration of the latter following the topical application of sunscreen in case of damaged or diseased skin; this phenomenon is favored by the nanometric dimensions of the particles of these oxides in the creams, which are adopted to avoid the undesired whitening effect of the creams themselves; in this regard, see for example the paper “Toxicity and penetration of TiO₂ nanoparticles in airless mice and porcine skin after subchronic dermal exposure”, Jianhong Wu et al., Toxicology letters 191 (2009) 1-8.

To overcome these problems, physical solar filters based on compounds other than those mentioned above have been proposed in recent years, in particular simple or modified hydroxyapatite-based materials.

Hydroxyapatite is the compound of formula Ca₅(PO₄)₃(OH), and is also referred to in the literature by the abbreviation HA, which will also be used in the present description. By “modified hydroxyapatite” in the present description we mean a HA in which part of the Ca²⁺, PO₄³⁻ or OH⁻ ions of the basic formula is replaced by other ions.

Patent EP 2410974 B1 discloses the use of nanostructured HA in a sunscreen, in combination with a chemical filter and a third component consisting of a metal salt of a carboxylic acid. According to this document, HA present in the sunscreen has preferably particle size in the range 1-200 nm, more preferably 5-95; HA of particle size in these ranges is said to allow a better dispersion of the hydroxyapatite in the cosmetic compositions wherein the sunscreen is used, thus significantly improving the distribution and absorption thereof on the skin upon application, while at the same time advantageously reducing the whitening effect. This document does not mention the source or the method of preparation of HA; moreover, the HA is not indicated as an active filter, but only as a booster of the sun protection effect (SPF, Sun Protection Factor) of the other components of the sunscreen.

The papers “Effect of Zn²⁺, Fe³⁺ and Cr³⁺ addition to hydroxyapatite for its application as an active constituent of sunscreens”, Journal of Physics: Conference Series 249 (2010), and “Phosphates nanoparticles doped with zinc and manganese for sunscreens”, Materials Chemistry and Physics 124 (2010) 1071-1076, both in the name of T. S. de Araujo et al., report the production of modified HA with partial substitution of calcium with the indicated ions, for use in sunscreens. In these two articles the preparation of the material is carried out synthetically, i.e. by co-precipitation starting from solutions containing, as precursor compounds of the modified HA specification, nitrates of calcium and of the other metals mentioned, and ammonium phosphate. This paper reports that a typical size range for physical sunscreens is 70-200 nm.

Patent application WO 2017/153888 A1 describes a synthesis route similar to that of the two cited articles by de Araujo et al.; the product obtained is a compound in which there is a simultaneous replacement of iron instead of calcium and titanium instead of phosphorus. In the conclusions, this paper reports that particles suitable for application as sunscreen have size lower than 120 nm.

The HA described in these documents is therefore obtained by chemical synthesis, which sometimes is not readily accepted by the cosmetic consumers; the use of green compounds and/or compounds of natural origin, on the other hand, is much more acceptable for the general public.

Patent application US 2017/0119636 A1 and the paper “A hydroxyapatite-Fe₂O₃ based material of natural origin as an active sunscreen filter”, C. Piccirillo et al., J. Mater. Chem. B, 2014, 2, 5999-6009, describe the production of mixtures comprising HA modified with partial substitution of the calcium ion with iron and Fe₂O₃ (hematite), produced from cod bones treated in solutions of iron ions and subsequently calcined. This preparative route avoids the use of specially produced chemical compounds, offering the advantage of using as raw materials the waste materials of the fishing industry; these sunscreens, however, have the disadvantage of having a red color, whose intensity depends on the ratio between the HA/hematite components, limiting the applications in final cosmetic products. The modified HA described in this document is in the form of powders with grain size essentially in the range 50-200 nm, with the majority of particles of size around 150 nm (see paragraph [0072]).

With regard to photoprotective compositions for plants, these in their turn generally contain physical sunscreens in the form of nanoparticles, or comprise chemicals and/or ingredients containing undesirable contaminants, such as lead, cadmium, fluoride, arsenic, aluminum and/or silicon.

Besides, some sunscreen formulations may employ synthetic ingredients to make the sunscreen formulation more hydrophobic.

A fundamental characteristic of UV-filters is their color, that has to be white so to avoid any alteration of the color of the final formulations and hence, once the sun cream is applied topically, of the color of the skin. This is a very important feature related to both consumers perception and safety, as an unnatural coloration of the skin is unpleasant, and more importantly can cover the effects of an excessive exposition to sun light, such as the formation of uprising sunburns.

It is therefore still felt in the field the need to have available sunscreens that are free from the problems outlined above, both for use in cosmetic compositions and for the photoprotection of plants.

The object of the present invention is to provide a material useful as a solar filter, as well as to provide a process for its production, and cosmetic or plant photoprotective compositions which comprise it.

### SUMMARY OF THE INVENTION

These objects are achieved with the present invention, which in a first aspect relates to a process for the production, starting from fish by-products, of hydroxyapatite or modified hydroxyapatite in the form of powders, optionally in combination with powders of tricalcium phosphate and of oxides of the elements used to modify hydroxyapatite, which comprises the following steps:
a) optionally, in case it is desired to produce a modified hydroxyapatite, immersion for a time between 15 minutes and 24 hours of fishbones in a solution containing ions of one or more elements selected among Zn, Ti, Mg, Mn, Sn, Se and Ag, and subsequent bone extraction from said solution;
b) drying of fishbones, pristine or deriving from step a, between 105-110° C. overnight;
c) placing of the fishbones, deriving from step b, in an open or ventilated oven in such an amount to have a ratio between the quantity of fishbones and the volume of the oven chamber equal to or smaller than 12 g per liter, and positioning the fishbones on a layer with a thickness equal or smaller than 1 cm;
d) treatment of fishbones in an oxidizing atmosphere at a temperature between 700° C. and 1000° C. for a time between 30 minutes and 8 hours so as to promote grain growth and coalescence obtaining particles larger than 250 nm;
e) after cooling to a temperature below 200° C., grinding of the product obtained from the heat treatment of step d with selection of the fraction of powders of size between 250 nm and 50 μm.

In a second aspect thereof, the invention relates to powders of hydroxyapatite or hydroxyapatite modified with one or more elements selected from Zn, Ti, Mg, Mn, Sn, Se and Ag in percentage by weight between 0.1 and 15%, possibly in combination with tricalcium phosphate and/or one or more oxides of said elements, having particles in the size range between 250 nm and 50 μm, and characterized by having white color, where this characteristic is defined as having CIELab coordinates in the following ranges: L in the range between +93.0 and +100.0, a in the range between −3.00 and +3.00, and b in the range between −3.00 and +3.00.

In a third aspect thereof, the invention relates to cosmetic sunscreen compositions comprising the powders described above.

Finally, in the last aspect thereof, the invention relates to UV-photoprotective compositions for plants comprising the powders described above.

### BRIEF DESCRIPTION OF THE FIGURES

FIG. 1 shows X-ray powders diffraction patterns of six samples of material of the invention, of which three have been obtained from sardine bones and three have been obtained from salmon bones treatment at different temperatures;

FIG. 2 shows scanning electron microscope (SEM) micrographs of various samples of materials of the invention obtained from sardine and salmon bones after treatment at different temperatures;

FIG. 3 shows the reflectance spectra of UV radiation of samples of materials of the invention and, by comparison, of zinc oxide;

FIG. 4 shows the absorption spectra of UV radiation of water/ethanol suspensions of materials of the invention and, by comparison, of zinc oxide and of an iron doped hydroxyapatite;

FIG. 5 shows the absorption spectra of water/ethanol suspensions of zinc oxide, of a material of the invention, and of a mixture thereof (labeled as Booster).

### DETAILED DESCRIPTION OF THE INVENTION

The invention is described below in detail with reference to the figures.

All percentages and concentrations indicated in the text, unless otherwise indicated, are by weight.

The materials produced and used in the invention are hydroxyapatite, of formula Ca₅(PO₄)₃(OH) (often also reported as its dimer Ca₁₀(PO₄)₆(OH)₂, which reflects the presence of two basic formula units in the elementary cell of the crystal), possibly in a modified form in which calcium is partly replaced by one or more elements selected from Zn, Ti, Mg, Mn, Sn, Se and Ag, and/or the phosphate and/or hydroxide ions are partly replaced by one or more oxyanions of the same elements, and possibly in mixture with tricalcium phosphate, Ca₃(PO₄)₂ and oxides of one or more of said elements. Tricalcium phosphate, when present, is generally in the form of its β polymorph, the one stable at lower temperatures: this compound also exists in the form of polymorphs a and a′, but for their formation higher temperatures are necessary than those of the process of the invention. Compound β-Ca₃(PO₄)₂ is also referred to in the literature by the abbreviation β-TCP, which will also be used in the present description. The term “material of the invention” will therefore be understood generically, unless otherwise specified, a mixture of hydroxyapatite, modified or not with one or more of the elements mentioned above, β-Ca₃(PO₄)₂, and possibly smaller amounts of one or more oxides of said elements; these one or more elements will also be referred to as “doping element(s)”. The amount of doping elements in the modified HAs of the invention can range between 0.1 and 15%.

In the process of the invention it is possible to use bones of fish essentially of any type, both of sea and fresh water, such as, sea bass, sea bream, amberjack, cod, tilapia, carp, and preferably oily fish, a broad class of fishes including mackerel, tuna, swordfish, trout, salmon, sardines, herring and anchovies.

Before carrying out step b, or a and b, the fishbones may optionally be, and preferably are, pre-cleaned from residues of organic tissues, for example by mechanical treatments or by treatment for a period of a few hours with hot water (e.g. at 80° C.) or with aqueous solutions of chemical agents, for example sodium hypochlorite; the so cleaned bones can then be used immediately, or dried and stored in preparation of the treatments to be carried out afterwards.

Optional step a is performed when it is desired to produce a modified HA. This step consists in immersing the fishbones for a time between 15 minutes and 24 hours and a temperature in the range between 4° C. and 80° C. in a solution containing ions of one or more doping elements selected from Zn, Ti, Mg, Mn, Sn, Se and Ag or mixtures thereof.

The solvent of the solution could be not completely aqueous, for instance a hydroalcoholic solution; however, an aqueous solution (i.e., in which water is the sole solvent) is normally and preferably employed. The solution is produced with compounds soluble at room temperature of one or more doping elements with which it is desired to modify the final HA; soluble compounds suitable for the purpose are, for example, salts or organometallic compounds of the cited elements. Among salts, acetates or salts of other organic acids, chlorides for most elements (except, for example, silver) and nitrates can be used; the preferred salts are nitrates and chlorides. The preferred organometallic compounds are the alkoxides of the cited elements. The solution preferably has concentration between 1 and 10 g/L, referring to the one or more doping element, and is used in such a volume as to completely cover the fishbones and such that the weight ratio between the total amount of doping element initially in solution and the bones is between 0.1 and 50%. The immersion of the bones in the solution of the element(s) compound(s) is prolonged for a time between 15 minutes and 24 hours, at a temperature between 4 and 80° C., preferably at room temperature. After this treatment, the bones are extracted from the solution for use in the next step.

Step b, that is the drying of fishbones, pristine or deriving from step a, at a temperature between 105-110° C. overnight, is carried out to reduce the water content of samples.

The following step, c, consists in positioning the fishbones inside an open or ventilated oven. The quantity of fishbones placed in the oven has to be proportional to the volume of the oven chamber, and such to keep the ratio between the quantity of fishbones and the volume of the oven not higher than 12 g per liter.

The material is placed in the oven in the form of a layer of thickness lower than 1 cm so to ensure the complete combustion of the organic matter inside and among the fishbones, and the obtainment of a white material. In case the fishbones are in the form of powder, the fraction smaller than 0.2 mm is separated by a sieve. In order to achieve the complete oxidation of the organic matter and obtain a white material, this fraction has to be placed in the oven chamber in the form of a layer of thickness lower than 0.5 cm.

The inventors have observed that laying the fishbones in beds having the thickness values indicated above, depending on the size of the bones or bone powders, is an essential condition to obtain final powders with the desired color (whiteness, defined by the CIELab coordinates reported above).

The subsequent step, d, consists in calcining the bones in the oven in an oxidizing atmosphere at a temperature between 700° C. and 1000° C. for a time between 30 minutes and 8 hours. This thermal treatment can be carried out under a static atmosphere or a flow of an oxidizing gas, generally air. During this thermal treatment, the fishbones undergo a structural rearrangement, consisting in the oxidation of the organic matter and in the coalescence of mineral particles into particles of grain size higher than 250 nm.

The inventors have observed that if the thermal treatment of step d is carried out at temperatures lower than 700° C. or higher than 1000° C., the UV absorbing properties of the resulting material worsen, leading to a less efficient sun screening effect.

Heating from the initial temperature, normally room temperature, to the selected final temperature, preferably takes place with a constant ramp, for example of 2° C./min, keeping at the final temperature for a time between 30 minutes and 8 hours, and finally cooling, natural or forced, at a temperature of 200° C. or lower, and preferably at room temperature.

After reaching the desired final cooling temperature, in step e the calcined bones are ground by any known method, for example manually (in a mortar using a pestle), with a ball mill, or the like. After grinding, the obtained powders are subjected to sieving with mechanical sieves, selecting the fraction of powders of size less than 50 μm. The mechanical sieves are commercially available and are generally indicated with the unit “mesh”: the fraction of powders with particle size less than 50 μm is collected by sieving the powders through a 270 mesh commercial sieve. This step helps to level out the powders and remove large aggregates; on the other end, the lower size limit of 250 nm is guaranteed by the thermal treatment the bones are subjected to.

Alternatively, the materials can be reduced in the form of powders with particle size less than 50 μm by micronization or air classification.

In its second aspect, the invention relates to the powders obtained as a result of the process described above, also referred to below as “material(s) of the invention”.

These powders are generally constituted, in the case in which optional step a of the process is not carried out, by a mixture of phases comprising HA and β-TCP, in different ratios according to the type of fish whose bones are used and calcination temperature; alternatively, in case step a has been carried out, these powders are generally formed by a mixture of phases comprising modified HA, β-TCP, and minor amounts of oxides of the doping elements used in step a.

The materials of the invention have the following average chemical composition:
between 25.0 and 44.0 wt % of calcium, preferably between 28 and 40 wt %, more preferably between 30 and 36 wt %;
between 14.0 and 22.0 wt % of phosphorus, preferably between 15 and 18 wt %;
when one or more doping elements among zinc, titanium, magnesium, manganese, tin, selenium and silver are present, the total amount of said doping elements is lower than 15 wt %.

In general, regardless of the type of bone used and whether the HA is modified or not, the β-TCP/HA ratio increases with increasing calcination temperature; similarly, also the amount of oxides of the doping elements as separate phases increases with increasing calcination temperature. Regarding the influence on the final product of the type of bone used, the inventors have observed for example that in the case of sardine bones, at all calcination temperatures up to 1200° C. the HA phase remains largely predominant (minimum quantity of about 85% by weight), while using salmon bones, β-TCP is obtained as the predominant phase already at the lowest tested temperature (for example, about 54% after treatment at 600° C.), and the β-TCP/HA ratio remains stable with increasing calcination temperature.

The chemical composition of the material changes with the type of fishbone used and the different calcination temperatures. In the case of salmon bones, the inventors have observed a progressive increase of calcium and phosphorous concentration inside the material with increasing calcination temperature. In the case of sardine bones, this increase was only observed moving from a calcination temperature of 600° C. to 700° C., while at temperature from 700° C. to 1000° C. the concentrations of calcium and phosphorous almost remain constant.

Regardless of the kind of fishbones used, the volume specific surface area (VS SA) of the powder obtained from the calcination decreases with increasing the calcination temperature. As an example, the VSSA of the powder obtained from the calcination of sardine bones at 600° C. has a VSSA of 20.13±3.10 m²/cm³, while that obtained at 1000° C. has a VSSA of 7.20±2.05 m²/cm³. Similarly, the VSSA values of the powders obtained from the calcination of salmon bones at 600° C. and 1000° C. are 21.12±3.52 m²/cm³ and 8.92±1.93 m²/cm³, respectively.

The materials of the invention are characterized by the color, where this characteristic is defined as having CIELab coordinates in the following ranges: L in the range between +93.0 to +100.0, a in the range between −3.00 to +3.00, and b in the range between −3.00 to +3.00. The achievement of these color coordinates is ensured by the positioning of the fishbones inside the oven as described in step b of the invention, namely by keeping the ratio between fishbones quantity and the oven volume lower than 12 g per liter, and by arranging fishbone in a layer thinner than 1.0 cm, and in case of fine fishbones particles with a size smaller than 0.2 mm, thinner than 0.5 cm.

In its third aspect, the invention relates to a cosmetic composition comprising at least one fluid vehicle and the material of the invention. The vehicle must be and remain fluid or at least spreadable by hand in the range of temperatures typical for application for these cosmetic compositions, approximately from about −20° C. for use in the mountains to about 40° C.; fluid vehicles having these characteristics are well-known to the skilled person in the field of cosmetic compositions.

In addition to these two main necessary components, the cosmetic composition preferably comprises one or more suitable cosmetic ingredients, which can be selected from a wide range of additives known in the field of the formulation of these compositions, among which, just to give some examples, other organic or inorganic UV filters, tanning agents, rheological additives, buffering agents, antimicrobial agents, anti-isothermal agents, antistatic agents, coloring agents, skin conditioning agents, preservative agents, covering agents, denaturing agents, depigmenting agents, detangling agents, emollient agents, emulsifying agents, film-forming agents and moisturizing agents; waterproofing components can also be added to make the composition resistant in case of immersion in water.

According to a preferred embodiment, said cosmetic composition further comprises at least one active ingredient selected from organic and inorganic UV filters, anti-aging agents and antioxidants.

According to one embodiment the cosmetic composition is a sunscreen product, an eye make-up product, a facial make-up product, a lip care product, a hair care product, a hair styling product, a nail care product, a hand care product, a skin care product, or a combination product thereof. According to another embodiment the material of the invention is associated with at least one active agent selected from pharmaceutically active agents, biologically active agents, disinfecting agents, preservatives, flavoring agents, surfactants, oils, fragrances, essential oils, and mixtures thereof.

In said cosmetic composition, the material of the invention is present in an amount between 0.5 to 50 wt %, based on the total weight of the cosmetic composition, preferably from 0.5 to 30 wt %, and more preferably from 0.5 to 20 wt %. An amount of material of the invention in the composition in this range ensures a good compromise between high UV-shielding ability and minimal whitening effect on the skin, depending on the required SPF level.

The cosmetic composition containing the material of the invention can be in the form of cream, gel, milk, spray, emulsion, lotion, protective mask, foundation, oil or other formulations known in the cosmetic field for application on the skin. Preferably, the cosmetic composition is in the form of an emulsion. Milks contain a high percentage of water, are easily spreadable but must be reapplied more often than other products. Creams have a greater adhesiveness and, being more difficult to spread, are generally used for the face; they are often greasy and for this reason they are not suitable for all skin types. The hydrophilic gels are more suitable for persons with oily skin because the vehicle in which the solar filter product is dispersed does not contain fatty substances that grease the skin.

Finally, in its last aspect, the invention relates to plant sunscreen composition comprising at least one fluid vehicle and the material of the invention. In addition to these two main necessary components, the plant sunscreen composition preferably comprises one or more of the following ingredients: (i) other organic and inorganic photoprotective agents; (ii) a wetting agent to reduce interfacial tensions, allow efficient mixing of the ingredients of the sunscreen formulation, and facilitate uniform coverage of the surfaces of plant tissues by the sunscreen formulation; (iii) a dispersion agent to preserve the state of the dispersion and prevent re-aggregation of the aqueous suspension; (iv) an emulsifier to stabilize the aqueous suspension; (v) a preservative and/or a biocide to reduce microorganism populations or prevent microorganisms from growing; and (vi) an effective concentration of particles for forming a film that reduces transmission of ultraviolet (UV), visible (VIS) and/or near infrared (NIR) radiation.

In said plant sunscreen composition, the material of the invention is present in an amount between 5 and 95%, and preferably between 5 and 80% on the total weight of the composition.

The invention will be further described by the following experimental part, including the description of the methods for carrying out the characterization tests, and the examples of production of various forms of material of the invention and measurement of their properties.

Methods and Instruments

Chemical Analysis

The content of Ca, doping elements and P in the produced samples was determined by inductively coupled plasma optical emission spectrometry (ICP-OES) with a Liberty 200 spectrometer (Agilent Technologies 5100 ICP-OES, Santa Clara, Calif.).

ICP assay solutions were prepared by dissolving 20 mg of sample in 50 mL of an aqueous solution of 2% HNO₃ (pur. analysis grade=65%) or 2% HCl (pur. analysis grade=37%) both sold from Sigma-Aldrich.

Diffractometric Analysis

The phase composition of each sample was determined by X-ray diffraction (XRD) with a D8 Advance diffractometer (Bruker, Karlsruhe, Germany) equipped with a Lynx-eye position-sensitive detector using Cu Kα radiation (λ=1.54178 Å) generated at 40 kV and 40 mA. The XRD spectra were recorded in the 20 field 10-60° with a step (20) of 0.02° and a counting time of 0.5 seconds.

SEM Analysis

The morphology of the samples was analyzed using a scanning electron microscope (SEM), (FE-SEM, Carl Zeiss Sigma NTS GmbH Oberkochen, Germany).

DLS Analysis

The hydrodynamic diameter distributions of the materials were measured by dynamic light scattering (DLS) on a Zetasizer Nano ZS (Malvern Ltd., Worcestershire, UK) and reported as z-average values. A stable suspension for each material was obtained by sonicating with a tip sonicator a solution obtained by dispersing an aliquot of each sample in bi-distilled water at the concentration of 1.0 mg/ml. The suspensions were placed in an ice bath to cool down the samples during the sonication, and were finally analyzed by DLS. Ten runs of 30 s were performed for each measurement and four measurements were carried out for each sample over the period of 1 hour.

Color Determination

The color of the samples was measured with a CM-700d spectrophotometer (Konica-Minolta, Japan), which was calibrated with a standard white plate with coordinates L=97.59, a*=0.07, b*=1.89. The data were expressed according to the CIELab system.

UV-Visible Reflectance and Absorbance

The UV-Vis reflectance spectra of the samples were obtained with a Cary Bio spectrophotometer (Varian, Palo Alto, USA) equipped with an integration sphere; the instrument was calibrated with a Spectralon standard (Labsphere SRS-99-010).

To have a direct estimation of the Sun Protection Factor (SPF) of a hypothetical sunscreen containing 20 wt % of material of the invention, the adsorption profiles were acquired using the method described in paper “Reliable and simple spectrophotometric determination of sun protection factor: A case study using organic UV filter-based sunscreen products”, S. I. Yang et al., Journal of cosmetic dermatology 17(3), 518-522 (2018), with some modifications.

Briefly, 200 mg of material of the invention were transferred to a 100 mL volumetric flask, diluted to volume with citrate buffer 0.1 M and pH 6.2 and sonicated for 5 min. A 5.0 mL aliquot was transferred to 50 mL volumetric flask and diluted to volume with a mixture of water/ethanol.

The absorbance of the obtained solutions was recorded by the same spectrophotometer used for the reflectance measurements working in the cuvette mode. The data recorded were used to estimate the in vitro SPF of sunscreens by the formula:

SPF_{spectrophotometric}=CF×Σ₂₉₀³²⁰EE(λ)×I(λ)×Abs(λ)+6.65 (eq. 1)

wherein:
EE (λ): erythemal effect spectrum;
I (λ): solar intensity spectrum;
Abs (λ): absorbance of sunscreen product;
CF: correction factor (=10);
6.65 is a correction coefficient.

The formula was derived by that proposed in the paper “Determinação do fator de proteção solar por espectrofotometria”, J. D. S. Mansur et al., An. Bras. Dermatol., 61, 121-124, 1986, and was conceived so that a standard sunscreen formulation containing 8% homosalate (3,3,5-trimethylcyclohexyl-2-hydroxybenzoate) presented a SPF value of 4.

The EE×I values are constants and were determined in the paper “A comparison of in vivo and in vitro testing of sunscreening formulas”, R. M. Sayre et al., Photochemistry and Photobiology 29(3), 559-566 (1979), so that their sum is 1.

Volume Specific Surface Area (vSSA)

Samples VSSA were measured at liquid nitrogen temperature (−196° C.) using Brunauer-Emmett-Teller (BET) mode with a CONTROL 750 (CE Instruments) apparatus. Samples were dried in air at 100° C. for 30 minutes before the analysis.

### Example 1

This example is about the preparation of HA-based materials starting from sardine and salmon bones; these include both materials according to the invention and materials not of the invention: the latter are materials produced with calcination of fishbones at a temperature lower than 700° C. or higher than 1000° C., and have been prepared for comparison purposes.

300 grams of sardine bones where soaked in 100 mL of hot water at 80° C. for 2 hours. The material was then dried overnight at 105° C. in an open oven. The so obtained material was placed inside an open oven having a volume of 25 L in the form of a layer with a thickness lower than 1 cm to be thermally treated under atmospheric conditions.

The thermal program used had a ramp of 2° C./min from room temperature up to 600° C., keeping at this temperature for 1 hour, then letting the system cool to room temperature.

The material recovered from the oven was ground in an agate mortar and sieved at 50 μm, to give a quantity of final product of 150 grams. This material is named in Table 1 below as SDnCaP-6.

The procedure depicted above was repeated with sardine bones and salmon bones with calcination temperatures varying between 600 and 1200° C. All the produced materials were obtained placing the fishbones inside the oven on a layer having a thickness lower than 1 cm (for fishbones larger than 0.2 mm) or lower than 0.5 cm (for fishbone powders with a size smaller than 0.2 mm). The samples obtained from sardine bones and salmon bones were from SDnCaP-6 to SDnCaP-12, and from SMnCaP-6 to SMnCaP-12, respectively, as reported in Table 1.

To obtain samples of modified HA, 300 grams of fishbones where washed in 100 mL of hot water at 80° C. for 2 hours. The material was then dried on paper and soaked for 2 hours in 200 mL of a solution, kept at 80° C., of a compound of the desired element.

In case of zinc doping, zinc nitrate (ZnNO₃), was used for preparing an aqueous solution. In case of titanium doping, fishbones were soaked for 2 hours in 200 mL of a solution of titanium isopropoxide (Ti(OCH(CH₃)₂)₄), in isopropanol, kept at 80° C. The concentrations of these solutions were such to provide a metal/bone weight ratio (g/g) in solution as reported in Table 1 below, in the last column.

The material was then dried overnight at 105° C., and placed in an open oven in the form of a layer with a thickness lower than 1 cm to be thermally treated under atmospheric condition.

The thermal program used had a ramp of 2° C./min up from room temperature to 1000° C., then steady for 2 hours before letting the system cool at room temperature.

The material recovered from the oven was ground in an agate mortar and sieved at 50 μm.

The procedure described above was repeated in different conditions, according to Table 1 below:

TABLE 1

| | | Calcination | | |
|---|---|---|---|---|
| | | Temperature | Doping | Metal/bone |
| Sample name | Fish | (° C.) | element | ratio (g/g) |
| SDnCaP-6 | Sardine | 600 | — | — |
| SDnCaP-7 | Sardine | 700 | — | — |
| SDnCaP-8 | Sardine | 800 | — | — |
| SDnCaP-9 | Sardine | 900 | — | — |
| SDnCaP-10 | Sardine | 1000 | — | — |
| SDnCaP-11 | Sardine | 1100 | — | — |
| SDnCaP-12 | Sardine | 1200 | — | — |
| SMnCaP-6 | Salmon | 600 | | |
| SMnCaP-7 | Salmon | 700 | — | — |
| SMnCaP-8 | Salmon | 800 | | |
| SMnCaP-9 | Salmon | 900 | — | — |
| SMnCaP-10 | Salmon | 1000 | — | — |
| SMnCaP-11 | Salmon | 1100 | — | — |
| SMnCaP-12 | Salmon | 1200 | — | — |
| SMnCaP-10Zn × 10 | Salmon | 1000 | Zn | 0.2 |
| SMnCaP-10Zn × 15 | Salmon | 1000 | Zn | 0.3 |
| SMnCaP-10Zn × 20 | Salmon | 1000 | Zn | 0.4 |
| SMnCaP-10Zn × 30 | Salmon | 1000 | Zn | 0.6 |
| SMnCaP-10Zn × 50 | Salmon | 1000 | Zn | 1.0 |

On some of the samples thus prepared, chemical analyses by ICP-OES were carried out. The results are reported in Table 2:

TABLE 2

| | Ca (%) | P (%) | Ca/P | Zn (%) | (Ca + Zn)/P |
|---|---|---|---|---|---|
| Samples | Av | Av | Av | Av | Av |
| SDnCaP-6 | 34.70 ± 1.2 | 16.72 ± 0.9 | 1.60 ± 0.02 | | |
| SDnCaP-7 | 38.52 ± 1.6 | 18.71 ± 1.0 | 1.59 ± 0.02 | | |
| SDnCaP-8 | 37.70 ± 1.3 | 18.66 ± 1.1 | 1.56 ± 0.03 | | |
| SDnCaP-9 | 38.83 ± 1.3 | 18.87 ± 0.9 | 1.59 ± 0.01 | — | — |
| SDnCaP-10 | 37.73 ± 1.0 | 18.34 ± 1.1 | 1.59 ± 0.02 | | |
| SMnCaP-6 | 35.45 ± 1.5 | 17.32 ± 1.2 | 1.58 ± 0.03 | | |
| SMnCaP-7 | 36.18 ± 1.0 | 18.21 ± 1.3 | 1.54 ± 0.03 | — | — |
| SMnCaP-8 | 39.66 ± 1.7 | 18.83 ± 1.3 | 1.63 ± 0.03 | — | — |
| SMnCaP-9 | 42.68 ± 1.9 | 19.89 ± 1.2 | 1.67 ± 0.02 | | — |
| SMnCaP-10 | 44.00 ± 2.1 | 21.30 ± 1.5 | 1.60 ± 0.02 | — | |
| SMnCaP-10Zn × 10 | 34.81 ± 1.2 | 18.37 ± 1.3 | 1.47 ± 0.03 | 3.75 ± 0.3 | 1.56 ± 0.02 |
| SMnCaP-10Zn × 15 | 34.18 ± 1.5 | 18.38 ± 1.4 | 1.44 ± 0.03 | 5.04 ± 0.5 | 1.57 ± 0.01 |
| SMnCaP-10Zn × 20 | 33.23 ± 1.1 | 18.13 ± 1.2 | 1.42 ± 0.02 | 6.08 ± 0.6 | 1.58 ± 0.03 |
| SMnCaP-10Zn × 50 | 35.18 ± 1.3 | 18.44 ± 1.2 | 1.47 ± 0.03 | 7.87 ± 05 | 1.68 ± 0.02 |

As can be noted by the data in the table, in general in the undoped samples the variability in the chemical composition is mainly due to the natural origin of the bones (kind of fish), and secondarily it depends on the temperature of treatment. In salmon and sardine bones treated at 700° C. and 900° C., the former shows higher contents of Ca and P and a Ca/P ratio closer to 1.67, which is the stoichiometric value of HA, compared to the latter. Generally speaking, the amount of Ca and P increased with increasing the calcination temperatures for both kind of fishbones. In case of modified HA (Zn was used as representative of other possible doping elements) a Ca/P ratio in the narrow range between 1.42 and 1.47 was found, with no correlation with the amount of zinc salt used as source of doping element. However, the amount of Zn inside the material, in terms of (Ca+Zn)/P ratio, increases proportionally with the amount of Zn²⁺ in the doping solution.

### Example 2

The color of some of the samples prepared in Example 1 was measured and the relative CIELab coordinates are reported in Table 3. The CIELab coordinates reported in Table 3 show that all materials have a white color, going from the bright white of the sample obtained by the calcination of salmon bones at 1000° C., to the tenuous white of that obtained at 600° C.

TABLE 3

| Sample | L | a | b |
|---|---|---|---|
| SMnCaP-6 | 94.32 ± 0.12 | 0.81 ± 0.11 | 2.99 ± 0.11 |
| SMnCaP-7 | 98.83 ± 0.07 | 1.32 ± 0.13 | 1.55 ± 0.12 |
| SMnCaP-8 | 98.97 ± 0.05 | 1.03 ± 0.09 | −0.82 ± 0.10 |
| SMnCaP-9 | 99.53 ± 0.07 | 1.01 ± 0.05 | −0.73 ± 0.09 |
| SMnCaP-10 | 99.82 ± 0.07 | 0.02 ± 0.07 | 0.01 ± 0.07 |
| SMnCaP-10* | 81.01 ± 0.11 | −3.20 ± 0.12 | 3.50 ± 0.15 |
| SMnCaP-10** | 89.11 ± 0.16 | 0.03 ± 0.17 | 1.10 ± 0.11 |

The samples identified in the table above with * and ** have been obtained, respectively, from fishbones in the form of powder with a size smaller than 0.2 mm, positioned in the oven in the form of a layer of 1 cm, and from fishbones larger than 0.2 mm positioned in the oven in the form of a layer thicker than 1 cm.

When more than 300 g of fishbones were placed in the oven with 25 L of volume, or when the fishbones were disposed in a layer thicker than 1 cm, the final material had a greyish color. As an example, the L CIELab coordinates measured on salmon bones disposed on a layer with a thickness of 1.2 cm and calcined at 800 and 1000° C. had much lower values than the other samples.

### Example 3

Some of the samples prepared in Example 1 were subjected to XRD analysis followed by Rietveld refinement of the obtained data to evaluate their phase composition. The results of these tests are summarized in Table 4 below:

TABLE 4

| | Phase composition (wt %) | | |
|---|---|---|---|
| Sample | HAp | β-TCP | ZnO |
| SDnCaP-6 | 98.9 ± 0.3 | — | |
| SDnCaP-7 | 94.7 ± 0.5 | 5.3 ± 0.3 | — |
| SDnCaP-8 | 95.4 ± 0.7 | 4.4 ± 0.4 | |
| SDnCaP-9 | 95.0 ± 0.7 | 5.0 ± 0.9 | — |
| SDnCaP-10 | 94.1 ± 0.9 | 5.9 ± 0.6 | |
| SMnCaP-6 | 46.2 ± 1.1 | 53.8 ± 1.5 | — |
| SMnCaP-7 | 44.1 ± 1.2 | 55.9 ± 1.3 | |
| SMnCaP-8 | 41.6 ± 1.0 | 58.6 ± 1.1 | — |
| SMnCaP-9 | 42.1 ± 1.2 | 57.9 ± 1.5 | |
| SMnCaP-10 | 43.9 ± 1.8 | 56.1 ± 1.3 | — |
| SMnCaP-10Zn × 20 | 45.4 ± 0.3 | 53.8 ± 0.3 | 0.8 ± 0.1 |
| SMnCaP-10Zn × 30 | 47.4 ± 0.2 | 51.1 ± 0.2 | 1.5 ± 0.1 |
| SMnCaP-10Zn × 50 | 37.7 ± 0.3 | 58.9 ± 0.2 | 3.5 ± 0.2 |

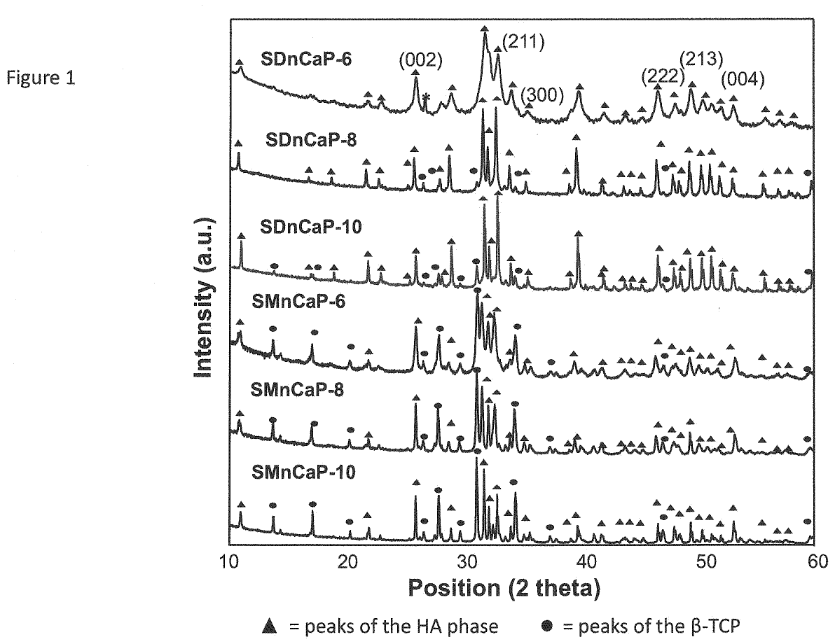

It can be observed from the data in the table that in case of undoped material from sardine bones, HA is always the predominant phase, with the amount of β-TCP increasing with the increasing of calcination temperature. This trend is visible in the three superimposed spectra reported in FIG. 1, in which the solid triangles indicate the peaks of HA phase, while the solid circles indicate the peaks of the β-TCP phase. In case of undoped material from salmon bones, the predominant phase is always β-TCP regardless of the calcination temperature, as can be observed from the spectra always reported in FIG. 1. Finally, in case of modified HA (from salmon bones, doped with zinc) the amount of ZnO detectable as a separate phase increases with increasing the treatment temperature.

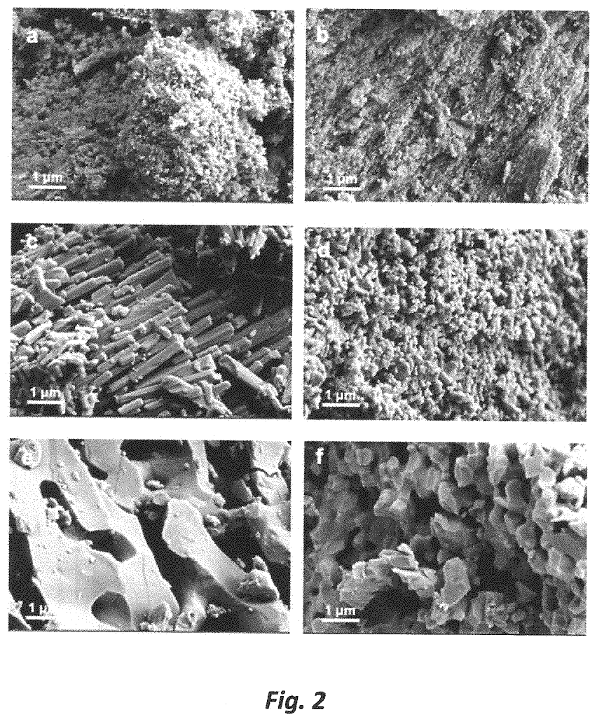

Some samples of Example 1 have also been studied by SEM to evaluate the trend of morphology depending on the calcination temperature. Some micrographs of samples obtained from different fishbones treated at different temperatures are reproduced in FIG. 2: in the figure, the three microphotographs on the left (a, c, e) are about materials obtained from sardines, while the three microphotographs on the right (b, d, f) are about materials obtained from salmons; pictures a and b are of samples obtained after treatment at 600° C., pictures c and d are of samples obtained after treatment at 900° C., and pictures e and f are of samples obtained after treatment at 1200° C. It can be seen from the microphotographs that at 600° C. the particles materials deriving from sardine and salmon are both of spherical shape and of similar size (few tens of nanometers). When the temperature is increased to 900° C., the size increases much more for the material obtained from sardine bones than for that obtained from salmon bones, while at 1200° C. both powders are sintered. The volume-specific surface area (VSSA) of the materials was measured by BET taking into account their density of 3.14 g/cm³. According to the EC's recommendation (European Commission 2011/696/UE) materials can be classified as nanomaterials if their VSSA is larger than 60 m²/cm³. The analysis revealed that bones treated at temperature less than 600° C. have VSSA values higher than 60 m²/cm³, and could be classified as nanomaterials. On the other hand, materials obtained from bones treated at temperature higher than 700° C. have generally VSSA values less than 42 m²/cm³, thus denoting their micrometric size.

### Example 4

The z-average values of the materials suspended in ultrapure water are reported in Table 5. As can be seen from the obtained values, the mean size of the primary particles ranges from 176 nm for the sardines bones treated at 600° C. to 755 nm for the sardines bones treated at 1000° C. The presence of particles of size lower than those observed by SEM is due to the fact that, as reported above, to have a good dispersion of the particles in water, the suspension is sonicated before the measure; this treatment could disaggregate the particles obtained from the process of the invention. A certain variability in the size of the materials due to the origin of fishbone could be observed; as an example, the z-average of the materials obtained from salmon bones were generally smaller compared to that obtained from sardine bones at the same temperature, with the mean size of the primary particles ranging from 132 nm for the salmon bones treated at 600° C. to 487 nm for those treated at 1000° C.

TABLE 5

| Samples | z-average (nm) |
|---|---|
| SDnCaP-6 | 176 ± 17 |
| SDnCaP-7 | 277 ± 29 |
| SDnCaP-8 | 361 ± 32 |
| SDnCaP-9 | 529 ± 50 |
| SDnCaP-10 | 755 ± 77 |
| SMnCaP-6 | 175 ± 20 |
| SMnCaP-7 | 266 ± 25 |
| SMnCaP-8 | 343 ± 34 |
| SMnCaP-9 | 447 ± 37 |
| SMnCaP-10 | 616 ± 55 |

### Example 5

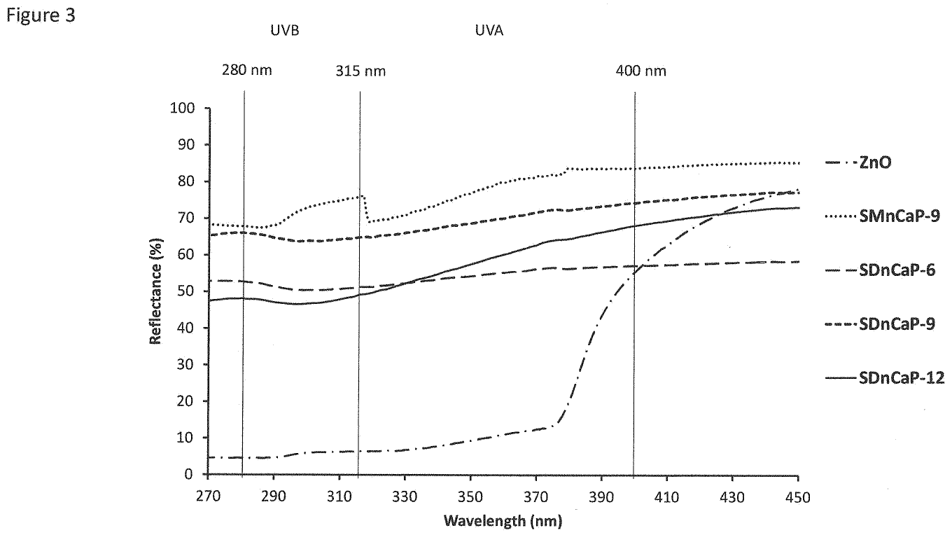

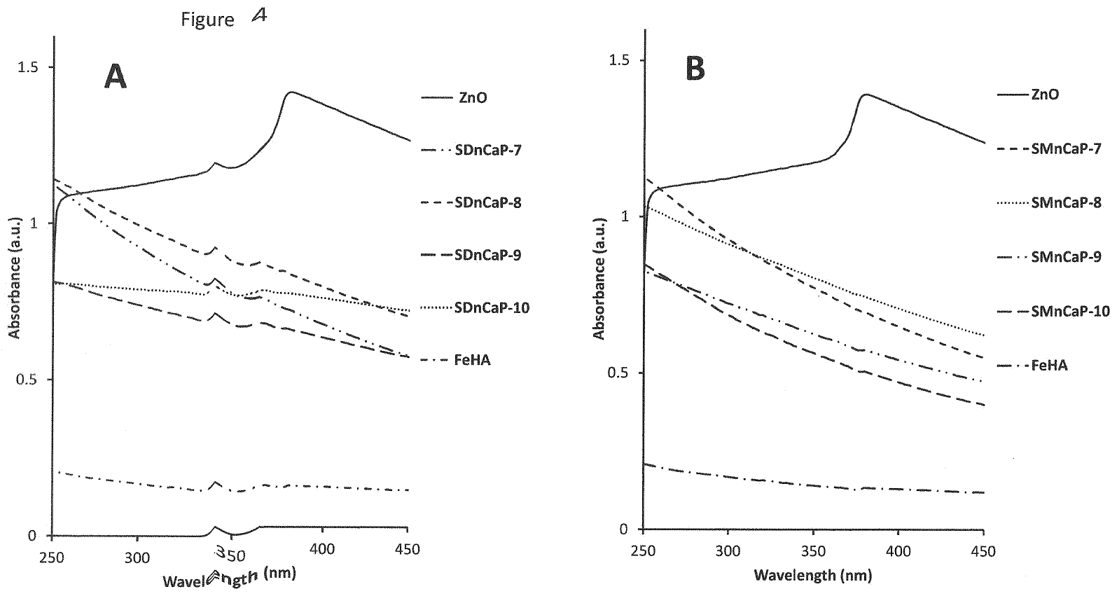

The reflectance and absorbance properties of some samples prepared in Example 1 were measured. The results of these tests are shown in FIG. 3, for the reflectance, and in FIG. 4 for the absorbance measures; in these figures, for comparison, analogous spectra of ZnO are reported.

As can be seen in FIG. 3, in general all samples of materials of the invention display a significantly higher reflectance compared to ZnO in the UVA-UVB region. More in detail, among all the reflectance spectra reported in this figure, those of un-doped salmon and sardine bones calcined at 900° C. have the higher values within these two windows.

FIG. 4 shows the UV absorption spectra of suspensions of ZnO, a sample of iron-doped HA, indicated as Fe-HA, produced according to the paper by M. Teixeira et al., Materials Science Engineering C, 71, 141 (2017); it is indicated as C15B M in the text, and of several samples of materials of the invention suspended in a mixture of water buffer and ethanol. The absorbance spectra of suspensions of samples obtained from sardine bones are reported on the left side of the figure, while those obtained from salmon bones are reported on the right one. These tests were carried out in order to assess and compare ZnO and Fe-HA photoprotective abilities with that of the materials of the invention, applying the method from Yang et al. described before to measure the in vitro SPF of a theoretical UV protective lotion obtained with the various tested materials. ZnO suspension spectrum displayed the highest adsorption values, with a peculiar shape featured by an absorption peak at 380 nm in the UVA region. The spectra of the materials of the invention all have a similar flat absorption profile, and all have much higher adsorption values compared to FeHA. The absorption intensities for sample SDnCaP-7 and sample SDnCaP-8 were the highest among those recorded. For samples treated at 900° C. and 1000° C. a decrease in the absorption values in the UVB region was observed, along with a lighter reduction in the UVA region, but the materials still showed good photoprotective property.

The trend observed for salmon bones treated at the different temperatures was the same of that described for sardines.

The SPF values calculated using equation (1) from the data of the spectra shown in FIG. 4 are reported in Table 6. As expected, ZnO solution displayed the highest SPF, with a value close to 18. The highest SPF value for materials of the invention was found for sample SDnCaP-8, which has a SPF value comparable to that of ZnO, followed by SDnCaP-10 and SDnCaP-9. For comparison, in the table is also reported the SPF value of FeHA. The SPF obtained from sardine bones at 600° C. was the lowest observed. A reduction of the SPF compared to those observed for materials calcined in the range between 700 and 1000° C. was observed for samples treated at 1100° C. and 1200° C., for which low UV absorption values were recorded (SPF in the range between 8.0 and 10.0). Once again, the trend observed for salmon bones treated at the different temperatures was the same to that described for sardines.

This implies that when the material is prepared at temperature below 700° C. or above 1000° C., the photoprotective performance of the material is strongly reduced regardless of the type of fishbone employed.

TABLE 6

| Samples | SPF in vitro |
|---|---|
| ZnO | 17.9 ± 0.8 |
| Fe-HA | 8.4 ± 0.3 |
| SDnCaP-6 | 7.6 ± 0.3 |
| SDnCaP-7 | 13.3 ± 0.4 |
| SDnCaP-8 | 16.5 ± 0.6 |
| SDnCaP-9 | 14.0 ± 0.5 |
| SDnCaP-10 | 14.5 ± 0.5 |
| SMnCaP-6 | 10.9 ± 0.3 |
| SMnCaP-7 | 13.7 ± 0.4 |
| SMnCaP-8 | 13.8 ± 0.5 |
| SMnCaP-9 | 13.5 ± 0.4 |
| SMnCaP-10 | 13.3 ± 0.3 |

### Example 6

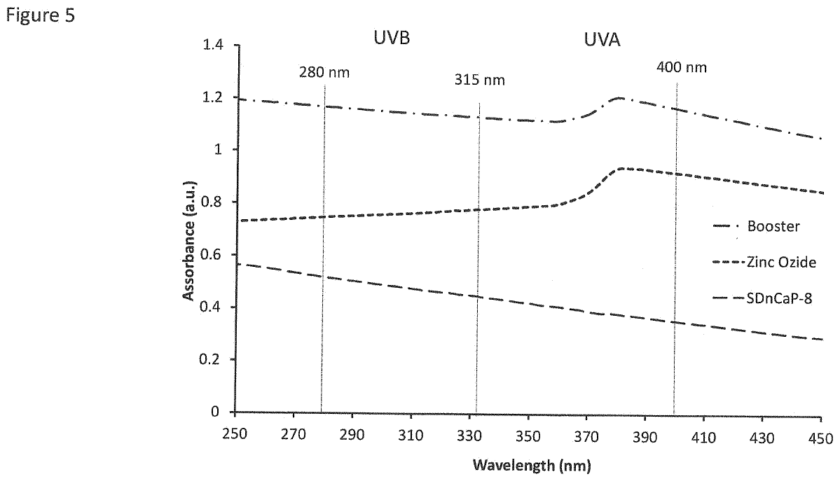

The SPF values of a suspension containing 100 ppm of ZnO and 100 ppm of SDnCaP-8 was measured, to assess the ability of the HA/β-TCP materials of the invention, obtained from a natural source, to boost the adsorption properties of ZnO. The relative spectra are reported in FIG. 5.

The mixture of ZnO and HA/β-TCP of the invention (labeled in FIG. 5 as Booster) was found to have the highest absorption value, with a relative SPF value of 18. It is to be noted that in this suspension the concentration of ZnO was half of the concentration of the ZnO suspension with SPF 17.9 (spectra in FIG. 5), as it was obtained mixing 1:1 a suspension of SDnCaP-8 at 200 ppm and a suspension of ZnO at 200 ppm.

### Comments to the Results

The results obtained in Examples 5 and 6 confirm that the materials of the invention are effective as physical solar filters when used alone, as well as boosters of the properties of commonly used physical filters, such as ZnO, and by extension as boosters of the properties of organic filters; these materials, obtained from recycle of waste products of the fishery industry, can be an effective alternative to known physical filters in the production of sunscreen compositions.

1. Process for the production of (A) hydroxyapatite in combination with tricalcium phosphate, or of (B) modified hydroxyapatite, in combination with tricalcium phosphate; and metal oxides of the elements used to modify hydroxyapatite, in the form of powders starting from fishbones, which comprises the following steps:
a) drying of fishbones between 105-110° C. overnight;
b) placing of the fishbones, deriving from step b, in an open or ventilated oven in such an amount to have a ratio between the quantity of fishbones and the volume of the oven chamber equal to or smaller than 12 g per liter, and positioning the fishbones on a layer with a thickness equal or smaller than 1 cm, with the proviso that any fraction of fishbones smaller than 0.2 mm is separated and placed in the oven chamber in the form of a layer of thickness lower than 0.5 cm;
c) treatment of fishbones in an oxidizing atmosphere at a temperature between 700° C. and 1000° C. for a time between 30 minutes and 8 hours so as to promote grain growth and coalescence obtaining particles larger than 250 nm;
d) after cooling to a temperature below 200° C., grinding of the product obtained from the heat treatment of step d with selection of the fraction of powders of size between 250 nm and 50 μm.

2. Process according to claim 1 in which the fishbones are pre-cleaned from residues of organic tissues by mechanical treatments or by treatment for a period of a few hours with hot water or with aqueous solutions of chemical agents.

3. The process according to claim 1, in which in optional step a said solution is obtained dissolving a soluble acetate, chloride, nitrate or organometallic compound of one or more of said elements, has a concentration between 1 and 10 g/L of said one or more elements, and is used in such a volume that the weight ratio between the total amount of said one or more elements initially in solution and the bones is between 0.1 and 50%.

4. The process according to claim 1, in which the selection of the fraction of powders of step e is carried out by sieving, air classification or micronization.

5. A mixture of hydroxyapatite and beta-tricalcium phosphate, in the form of powders, in combination with one or more oxides of one or more elements selected from Zn, Ti, Mg, Mn, Sn, Se and Ag, comprising:
between 25.0 and 44.0 wt % of calcium, preferably between 28 and 40 wt %, more preferably between 30 and 36 wt %
between 14.0 and 22.0 wt % of phosphorus, preferably between 15 and 18 wt %; and
a content of zinc, titanium, magnesium, manganese, tin, selenium and silver lower than 15 wt %;
characterized by:
a volume-specific surface area lower than 42 m2/cm3;
CIELab coordinates in the following ranges: L between +93.0 and +100.0, a between −3.00 and +3.00, and b between −3.00 to +3.00; and
particle size between 250 nm and 50 μm.

6. The cosmetic photoprotective composition comprising between 0.5 and 50 wt % of powders of claim 5 dispersed in a vehicle that is fluid in the range of temperatures between −20° C. and 40° C.

7. The cosmetic photoprotective composition according to claim 6, further comprising one or more cosmetic ingredients selected among organic or inorganic UV filters, tanning agents, rheological additives, buffering agents, antimicrobial agents, anti-isothermal agents, antistatic agents, coloring agents, skin conditioning agents, preservative agents, covering agents, denaturing agents, depigmenting agents, detangling agents, emollient agents, emulsifying agents, film-forming agents, moisturizing agents and waterproofing components.

8. The cosmetic photoprotective composition according to claim 6, further comprising at least one active ingredient selected from anti-aging agents and antioxidants.

9. The cosmetic composition of claim 6, wherein the cosmetic composition is a sunscreen product, an eye make-up product, a facial make up product, a lip care product, a hair care product, a hair styling product, a nail care product, a hand care product, a skin care product, or a combination product thereof.

10. The cosmetic composition of claim 6, wherein the powders of claim 5 is associated with at least one active agent selected from pharmaceutically active agents, biologically active agents, disinfecting agents, preservatives, flavoring agents, surfactants, oils, fragrances, essential oils, and mixtures thereof.

11. The use of the powders of claim 5 for boosting the sun protection factor (SPF) of a cosmetic composition having UV-A and/or UV-B protection and comprising at least one inorganic or organic UV filter and mixtures thereof.

12. Photoprotective composition for plants comprising between 0.5 and 95 wt % of powders of claim 5 dispersed in a vehicle that is fluid in the range of temperatures between 0° C. and 40° C.

13. Photoprotective composition for plants according to claim 12 further comprising at least one component selected among wetting agents, dispersion agents, emulsifier agents, preservative and/or biocide agents and particles for forming a film on plants reducing the transmission of ultraviolet, visible and/or near infrared radiation.

14. The mixture of hydroxyapatite and beta-tricalcium according to claim 5 wherein the hydroxyapatite by the following process:
a) immersion for a time between 15 minutes and 24 hours of fishbones in a solution containing ions of one or more elements selected among Zn, Ti, Mg, Mn, Sn, Se and Ag, and subsequent bone extraction from said solution;
b) drying of fishbones, pristine or deriving from step a, between 105-110° C. overnight;
c) placing of the fishbones, deriving from step b, in an open or ventilated oven in such an amount to have a ratio between the quantity of fishbones and the volume of the oven chamber equal to or smaller than 12 g per liter, and positioning the fishbones on a layer with a thickness equal or smaller than 1 cm, with the proviso that any fraction of fishbones smaller than 0.2 mm is separated and placed in the oven chamber in the form of a layer of thickness lower than 0.5 cm;
d) treatment of fishbones in an oxidizing atmosphere at a temperature between 700° C. and 1000° C. for a time between 30 minutes and 8 hours so as to promote grain growth and coalescence obtaining particles larger than 250 nm;
e) after cooling to a temperature below 200° C., grinding of the product obtained from the heat treatment of step d with selection of the fraction of powders of size between 250 nm and 50 μm.

15. The process for the production of hydroxyapatite in combination with tricalcium phosphate, or of modified hydroxyapatite in combination with tricalcium phosphate and metal oxides of the elements used to modify hydroxyapatite, in the form of powders starting from fishbones, which comprises, before the step of a) drying of fishbones, the step of:
a1) immersion for a time between 15 minutes and 24 hours of fishbones in a solution containing ions of one or more elements selected among Zn, Ti, Mg, Mn, Sn, Se and Ag, and subsequent bone extraction from said solution.
